(19) United States
(12) Patent Application Publication
(10) Pub. No.: US 2023/0010191 A1
(43) Pub. Date: Jan. 12, 2023

(54) FASCIA TISSUE TREATMENT DEVICE WITH ATTACHMENTS FOR ENHANCED TREATMENTS

(71) Applicant: Ashley Diana Black International Holdings, LLC, Friendswood, TX (US)

(72) Inventor: Ashley Diana Black, Friendswood, TX (US)

(21) Appl. No.: 17/374,835

(22) Filed: Jul. 13, 2021

Related U.S. Application Data
(60) Provisional application No. 63/220,967, filed on Jul. 12, 2021.

(51) Int. Cl. A61H 11/00

(52) U.S. Cl. CPC A61H 11/00; A61H 2201/10; A61H 2201/0188; A61H 2201/1692; A61H 2201/1695

(57) ABSTRACT

A fascia tissue treatment device includes a bar member, at least one tissue treatment element supported by the bar member, and at least one treatment accessory removably connected to the bar member.

### CROSS-REFERENCE TO RELATED APPLICATIONS

This application is claims the benefit of U.S. Provisional Patent Application No. 63/220,967, filed Jul. 12, 2021, the contents of which are hereby incorporated by reference in their entirety.

### BACKGROUND OF THE INVENTION

Fascia tissue is a layer of fibrous tissue that operates as a connective tissue that surrounds muscles, groups of muscles, nerves, blood vessels, etc. The tissue allows for proper functioning of muscles with respect to one another (e.g., sliding past one another). When fascia tissue becomes damaged through injury, tissue knots, medical reasons, or otherwise, the fascia tissue can take time to correct itself or require manipulation to release the fascia tissue and allow for proper functioning of the tissue to allow the underlying muscle to properly operate. In some cases, the fascia tissue can be released or corrected without much difficulty, while in other cases, restoring the fascia tissue to its proper form can take considerably more effort. Other reasons for releasing fascia tissue may include cosmetic reasons, especially for people who have dimpled skin, which is often caused by fascia tissue extending through fat cells, thus causing dimples to appear on the skin. Wrinkled skin is also often affected by damaged fascia tissue. Distorted fascia tissue and adhesions of the fascia tissue may also lead to pinched nerves, reduced circulation, and other problems. Often, when the fascia tissue is properly released, the dimples can be considerably reduced or eliminated.

### BRIEF DESCRIPTION OF THE FIGURES

FIG. 1 is an illustration of a top view of an illustrative fascia tissue treatment device with an illustrative treatment accessory;

FIG. 2 is an illustration of a bottom isometric view of the illustrative fascia tissue treatment device and illustrative treatment accessory of FIG. 1;

FIG. 3 is an illustration of a bottom isometric view of the illustrative treatment accessory of FIG. 1;

FIG. 4 is an illustration of a top view of an illustrative fascia tissue treatment device with an illustrative treatment accessory;

FIG. 5 is an illustration of a bottom isometric view of the illustrative fascia tissue treatment device and illustrative treatment accessory of FIG. 4;

FIG. 6 is an illustration of a bottom isometric view of the illustrative treatment accessory of FIG. 4;

FIG. 7 is an illustration of a top view of an illustrative fascia tissue treatment device with an illustrative treatment accessory;

FIG. 8 is an illustration of a top isometric view of an illustrative fascia tissue treatment device;

FIG. 9 is an illustration of a top view of an illustrative fascia tissue treatment device with an illustrative treatment accessory;

FIG. 10 is an illustration of a top view of an illustrative fascia tissue treatment device with an illustrative treatment accessory;

FIG. 11 is an illustration of a bottom view of the illustrative fascia tissue treatment device with the illustrative treatment accessory of FIG. 10;

FIG. 12 is an illustration of a side view of the illustrative fascia tissue treatment device with the illustrative treatment accessory of FIG. 10;

FIG. 13 is an illustration of a top view of an illustrative fascia tissue treatment device;

FIG. 14 is an illustration of a bottom view of the illustrative fascia tissue treatment device of FIG. 13;

FIG. 15 is an illustration of a cross-sectional view of an illustrative fascia tissue treatment device with an illustrative treatment accessory;

FIG. 16 is an illustration of a bottom isometric view of an illustrative fascia tissue treatment device shown with an illustrative treatment accessory being positioned in a first orientation;

FIG. 17 is an illustration of a bottom isometric view of the illustrative fascia tissue treatment device of FIG. 16, shown with the illustrative treatment accessory being positioned in a second orientation;

FIG. 18 is a schematic illustration of an illustrative tissue treatment system; and

FIG. 19 is a schematic illustration of an illustrative tissue treatment system.

### DETAILED DESCRIPTION OF THE DRAWINGS

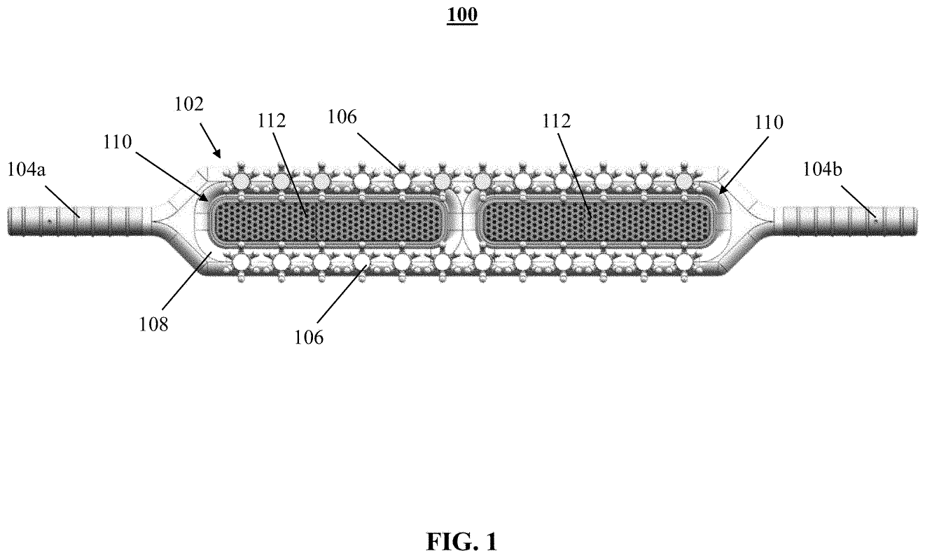

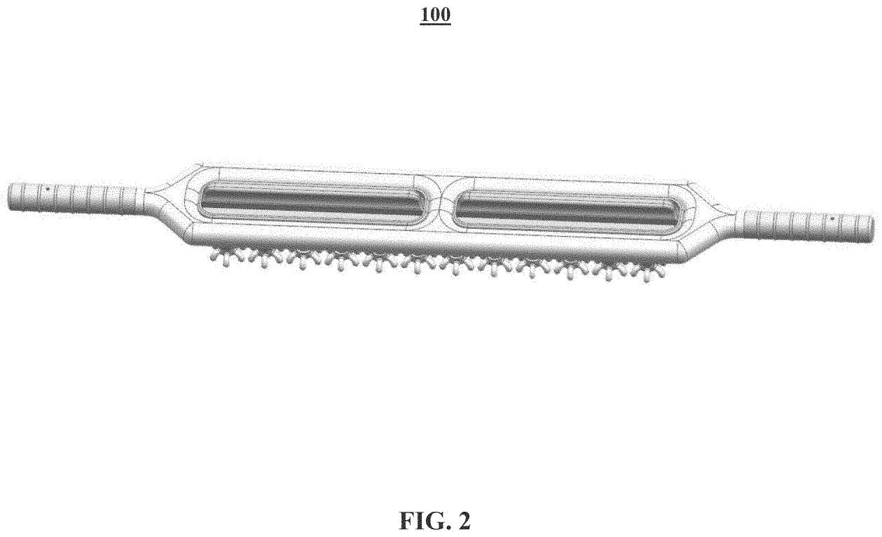

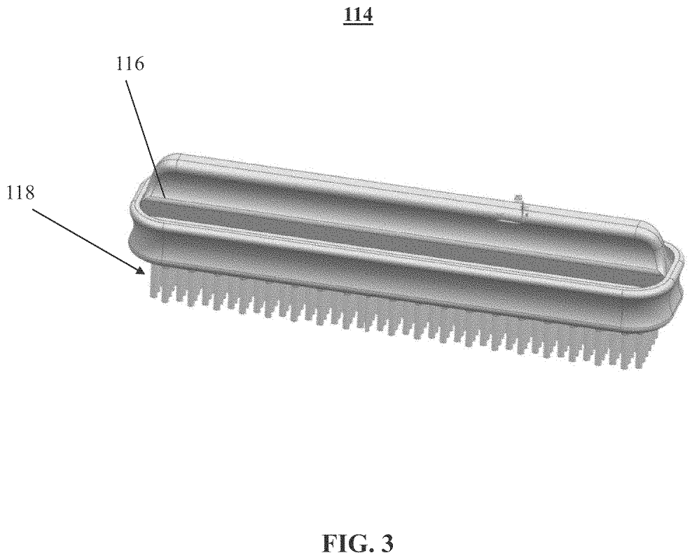

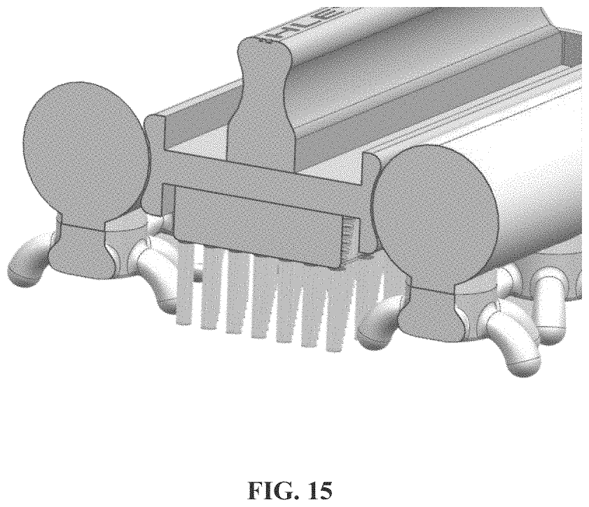

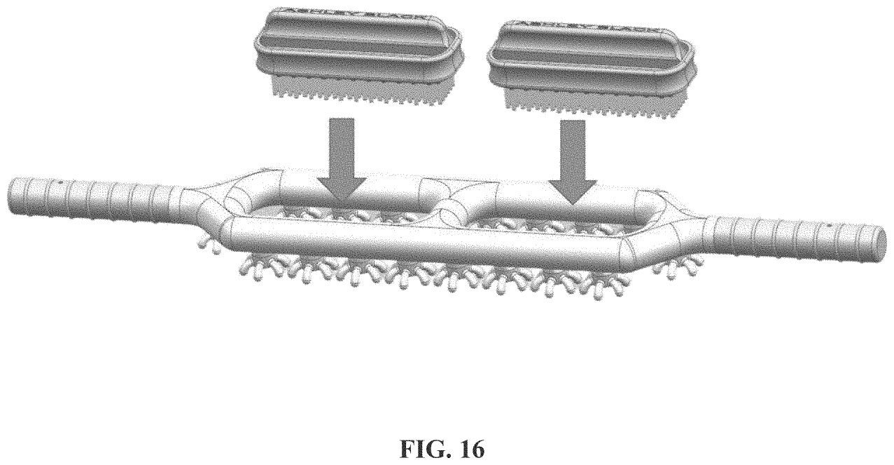

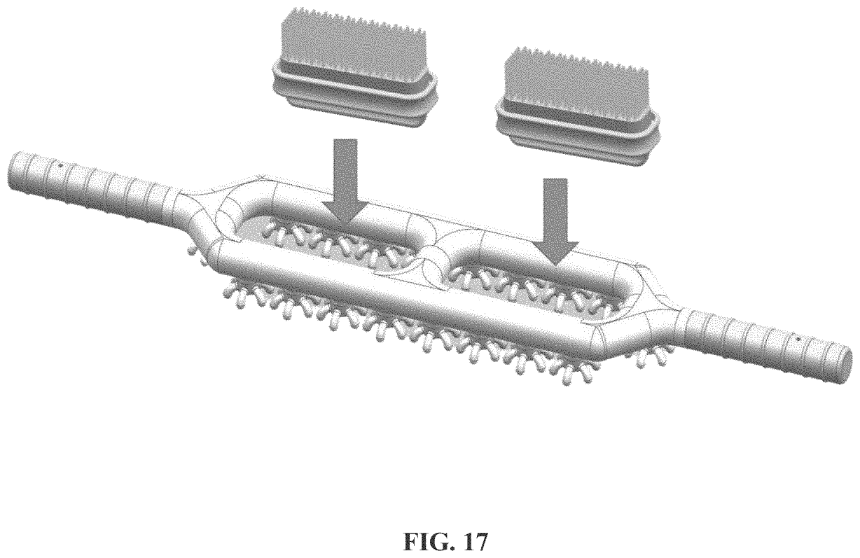

With regards to FIGS. 1-2, an illustrative fascia tissue treatment device 100 (hereinafter “treatment device 100”) is shown. In an embodiment, the treatment device 100 includes bar member 102, handle portions 104a, 104b coupled to the bar member 102, and a plurality of treatment elements 106 extending from the bar member 102. Bar member 102 is formed of a frame portion 108 which surrounds one or more apertures 110 in the bar member 102. The apertures 110 may be configured to receive a treatment accessory 112. A treatment accessory may be any device that is used to complement fascia tissue treatment by the treatment elements 106, but are not themselves treatment elements. For example, and as further described herein, the treatment accessory 112 may include LEDs to output certain wavelengths and electrical stimulation devices to output certain electrical currents to stimulate fascia and/or muscle stimulation to enhance fascia tissue treatment by the fascia tissue treatment device 100. The illustrative treatment device 100 of FIGS. 1-2 may specifically include the following illustrative features:
Total length of the bar member 102 and handle portions 104a, 104b may be between 25-35 inches. In an embodiment, the total length is 31 inches.
Twenty-four (24) treatment elements 106 may be arranged in two rows on the bar member 102.
The frame portion 108 may form two apertures 110 in the bar member 102.
One treatment accessory 112 may be received and retained in each of the two apertures 110.
   The treatment accessory 112 is a brush 114 having a base portion 116 that engages with the frame portion 110 of the bar member 102 and bristles 118 extending from the base portion 116, as shown in FIG. 3.
   Brush 114 may be approximately 8 inches in length.
   The base portion 116 may be made of a rigid material surrounded by a soft, flexible material such as plastic, rubber, or silicone in order to engage in a friction-fit relationship with the frame portion 108 of the bar member 102, as shown in FIG. 15.
As further depicted in FIG. 15, the frame portion 108 may have a circular cross section shape.
The treatment accessory 112 may be received in the aperture 110 in various arrangements, as depicted in FIGS. 16-17.
   Specifically, the bristles 118 of the brush 114 may be arranged to extend in the same direction as the treatment elements 106 (FIG. 16), or in the opposite direction as the treatment elements 106 (FIG. 17).
   The bristles may have a wide range of lengths, thicknesses, materials, and other dimensions depending on the function the brushes are to perform. For example, long and soft bristles may be used to relax the skin, while more rigid bristles with sharper tips may be used to perform exfoliation during use of the device. In an embodiment, the base portion 116 may be longer in the bristle direction, thereby enabling the bristles to be shorter and more rigid during treatment. Conversely, the base portion 116 may be shorter in the bristle direction, thereby enabling the bristles to be longer and less rigid during treatment.
   The shape of the base portion 116 has a grip portion extending from the base portion 116 on the opposite side of the bristles 118 that enables a user to use the brush 114 independent of the device 100. It should be understood that the grip portion may have a variety of different shapes and materials. In an embodiment, no grip portion may be included.

It should be understood that the features of the illustrative treatment device 100 of FIGS. 1-2 are examples, and that a wide range of alternative configurations may be possible to provide a fascia tissue treatment device with additional attachment(s) for enhanced treatments. For example, the handle may be shorter or longer, number of rows of treatment elements 106 may be one or more, number of treatment elements 106 may be fewer or more, number of apertures 110 may be one or more, treatment accessory 112 may be other than a brush, where the treatment accessory 112 may be longer or shorter. Moreover, the shape of the treatment elements 106 may be different. For example, rather than the treatment elements 106 being in the shape of a claw or flower, the treatment elements 106 may be in any other configuration.

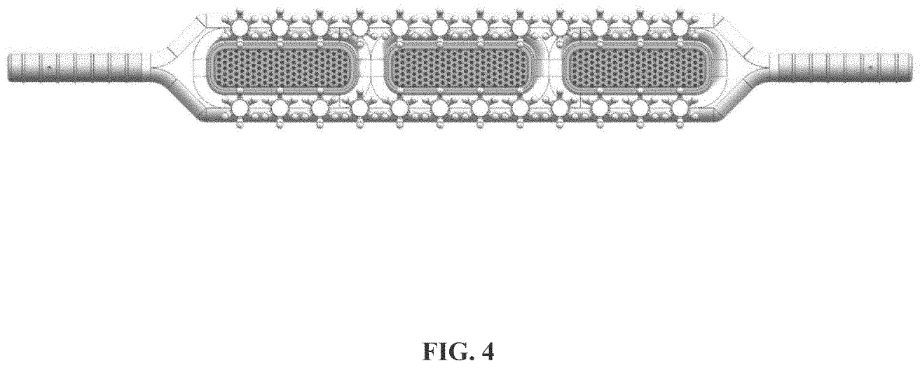

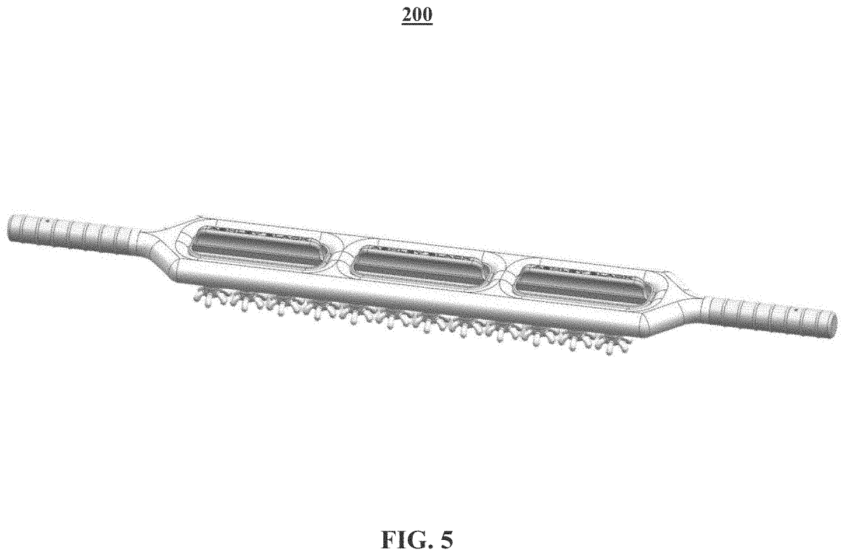

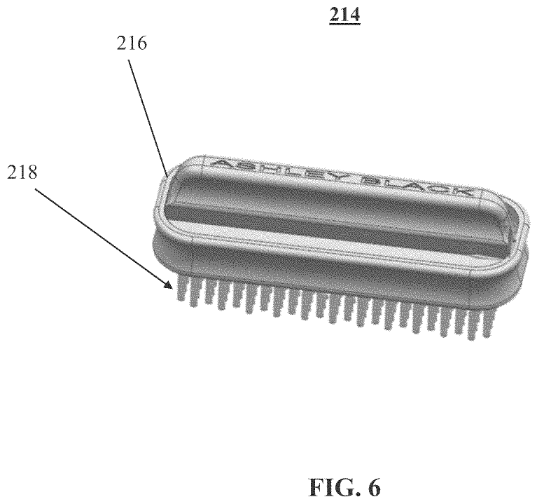

With regards to FIGS. 4-5, another illustrative fascia tissue treatment device 200 (hereinafter “treatment device 200”) is shown, which is similar to treatment device 100. The illustrative treatment device 200 of FIGS. 4-5 specifically includes the following illustrative features:
Total length of the bar member and handle portions is between 25-35 inches. In an embodiment, the total length may be 31 inches.
Twenty-four (24) treatment elements arranged in two rows on the bar member.
The frame portion forms three apertures in the bar member.
One treatment accessory is received in each of the three apertures.
   The treatment accessory is a brush 214 having a base portion 216 which engages with the frame portion of the bar member and bristles 218 extending from the base portion, as shown in FIG. 6.
   Brush 214 is approximately 5 inches in length.
   The base portion 216 is made of a rigid material surrounded by a soft, flexible material such as plastic, rubber, or silicone in order to engage in a friction-fit relationship with the frame portion of the bar member, as shown in FIG. 15.
As further depicted in FIG. 15, the frame portion may have a circular cross section shape.
The treatment accessory may be received in the aperture in various arrangements, as depicted in FIGS. 16-17.
   Specifically, the bristles of the brush may be arranged to extend in the same direction as the treatment elements (FIG. 16), or in the opposite direction as the treatment elements (FIG. 17). The bristles and base portion may be the same or similar to those described hereinabove.

In the illustrative embodiments shown, the treatment elements (i.e., treatment elements 106) have a flower-like appearance including a base portion coupled to the bar member and a plurality of finger members extending from the base portion. The treatment elements are also shown to be a single member (i.e., formed as a single element from a single piece of material). However, treatment elements may be formed of multiple elements.

Furthermore, rather than the device using treatment elements that have a flower-like appearance (i.e., central portion with extending finger members), treatment elements with non-flower-like appearance may be utilized, as well, that still provides a user with a number of closely spaced pressure-point elements that can be pressed and guided along a person's skin to cause fascia tissue to be released or perform a non-therapeutic function. For example, the treatment elements may include any rigid treatment feature which may be curved and have preferably round or bulbous tips. However, straight treatment features may be utilized, as well. The treatment features may be sized and configured such that the skin-contacting tips of the treatment device are substantially co-planar so that a pressure load applied to the skin and fascia tissue is substantially equally applied by each of the treatment features.

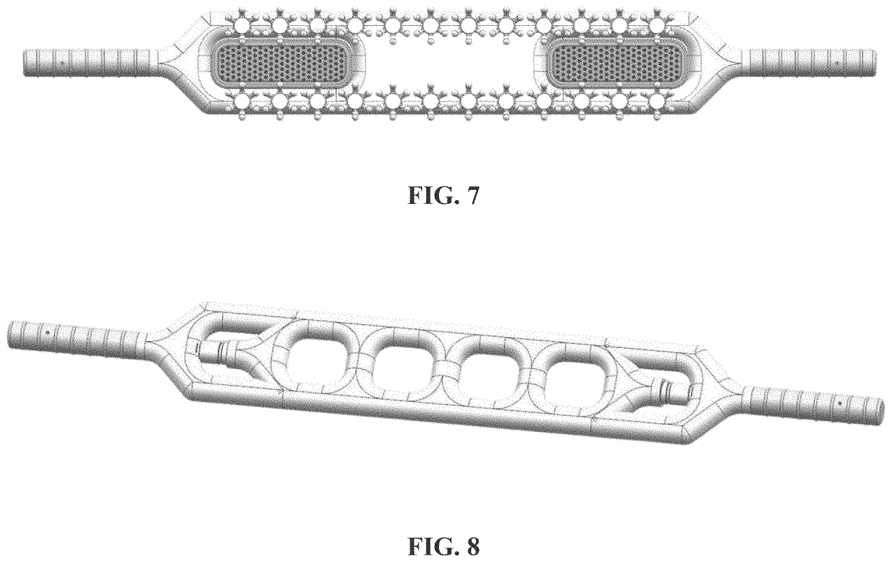

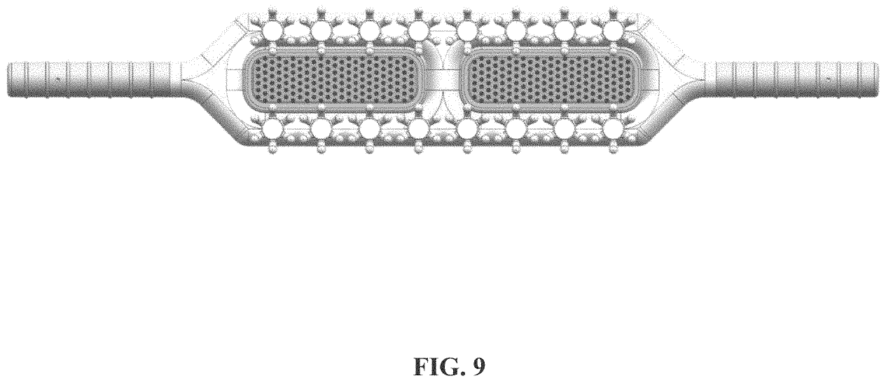

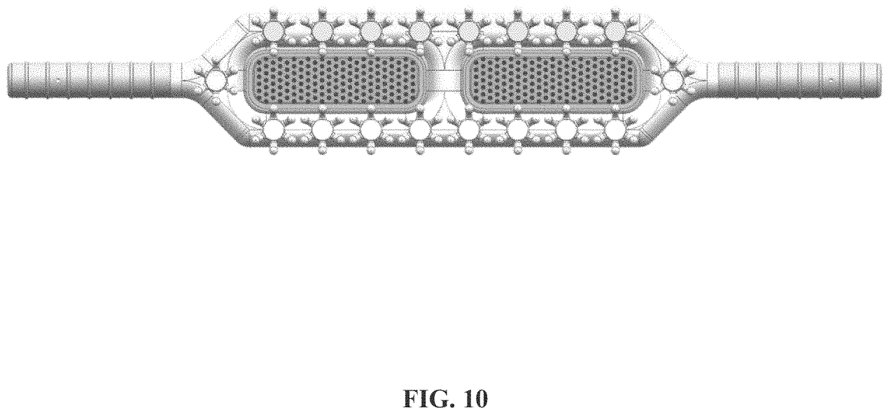

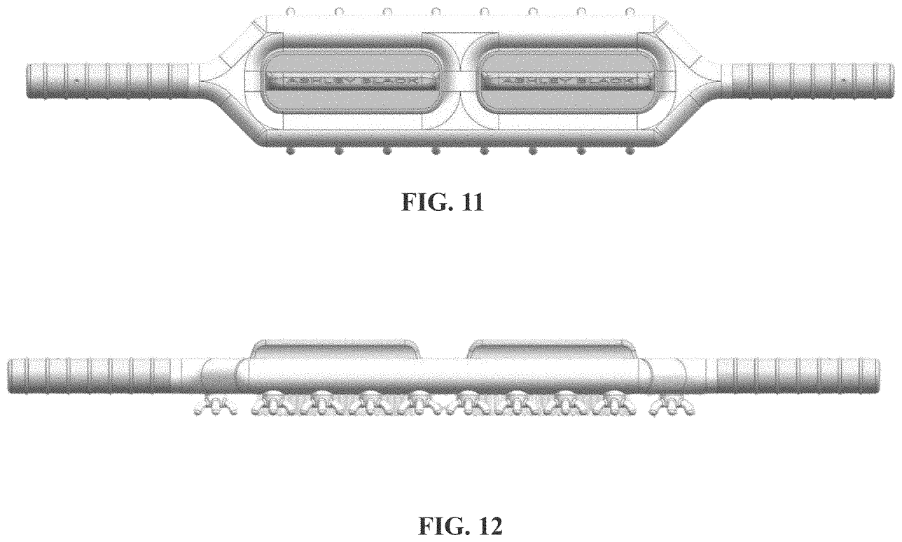

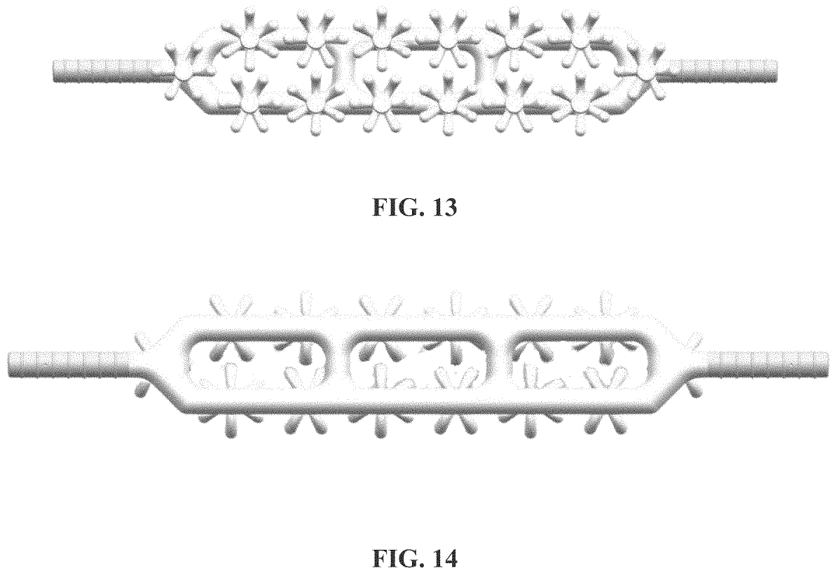

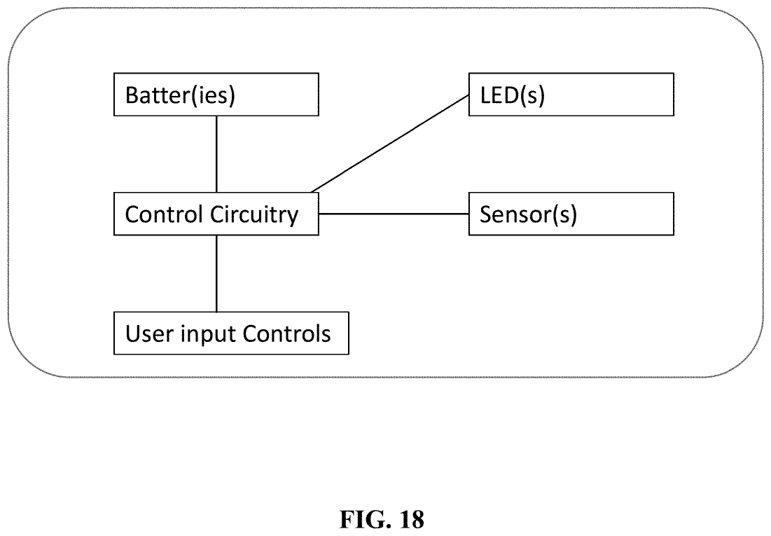

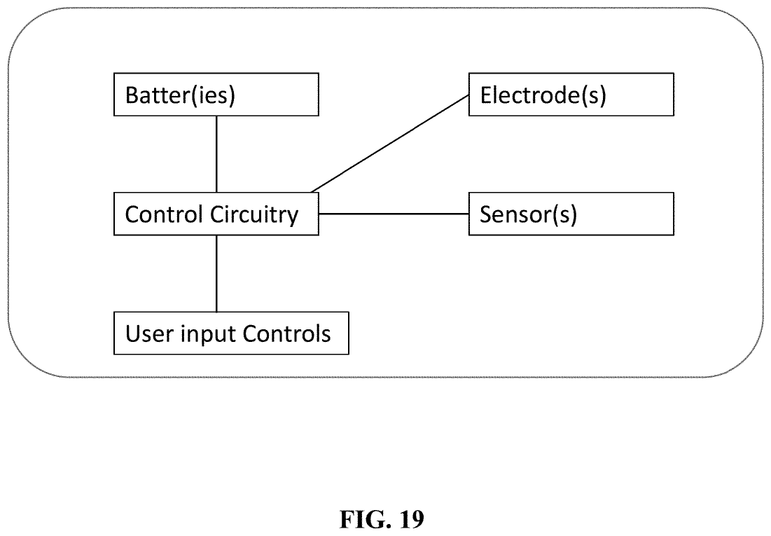

In other illustrative embodiments, a fascia tissue treatment device may be similar to the treatment device 100 of FIGS. 1-2 and/or treatment device 200 of FIGS. 3-4, but may alternatively or additionally include:
A bar member with no handles or different types of handles attached at the ends or other area of the bar member. In such embodiments, the device may include additional features for positioning and/or securing the device to a user's body.
A different total length of the bar member and handles (if present), as depicted in the illustrative examples of FIGS. 9-14.
A different number, shape, size, or arrangement of apertures, as depicted in the illustrative examples of FIGS. 7-14.
Apertures that do not receive a treatment accessory, as depicted in the illustrative examples of FIGS. 8, 13, and 14. In such an embodiment, forming apertures in the bar member requires less material and make the treatment device lighter in weight for ease of use.
A different number, shape, size, or arrangement of treatment elements, as depicted in the illustrative examples of FIGS. 8-14. For example, FIG. 8 includes zero treatment elements. FIG. 9 includes sixteen treatment elements arranged in two rows on the bar member. FIGS. 10 and 12 include eighteen treatment elements, with sixteen being arranged in two rows on the bar member and two additional treatment elements at each end of the bar member near the handle portions. FIGS. 13-14 includes fourteen treatment elements, with twelve being arranged in two rows on the bar member and two additional treatment elements at each end of the bar member near the handle portions.
The frame portion of the bar member may have any cross-sectional shape including hexagonal, pentagonal, square, oval, or the circular cross-sectional shape (tubular member), as shown in FIG. 15.
Alternative mechanism(s) to hold, secure, or otherwise engage the treatment accessory with the bar member. For example, instead of a friction fit between the treatment accessory and the frame portion of the bar member, a securing mechanism such as a pin, hook, latch, screw, fastener, fastening material (i.e., hook and loop fastening material), etc., may be used to secure the treatment accessory to the bar member. Alternatively, a base portion of the treatment accessory may remain integral with the bar member, while a face of a treatment accessory is removable and/or replaceable. For example, a treatment accessory face comprising a plurality of bristles may be removably coupled to a base portion that is integral with the bar member.
The bristles of the brush treatment accessory may be made of any suitable material including nylon, polypropylene, horse hair, feather, or any other natural or synthetic filament material. The tips of the bristles may be flocked (split) or unflocked. The tips of the bristles may also be rounded, bulbous, flat, pointed, etc. The bristles may be soft and flexible for a comfortable and soothing treatment, or may be rigid and stiff for a more aggressive tissue treatment. In some embodiments, the bristles include both soft/flexible bristles and rigid/stiff bristles for a combined treatment. In other illustrative embodiments, a first brush of the treatment device may have a first type of bristles and a second brush having a different type of bristles.
A light therapy system including a plurality of LED lights directed toward the skin to provide additional therapy to the fascia tissue. The light therapy system may provide light in one or more of the following forms: Red light (625 nm), Blue light (415 nm), Red+Blue light (625 nm˜415 nm), Infrared (760 nm). Batteries and control circuitry may also be included within the treatment accessories 112, for example, for powering and controlling operation of the LEDs (see, for example, FIG. 18). User input controls, such as knobs, buttons, or otherwise, may be accessible to a user to control operation of the LEDs and control circuitry. In an illustrative embodiment, the LEDs are positioned on the bar member, the treatment elements, and/or the treatment accessories in any location in which the light can be properly directed to the skin (i.e., is not blocked by another element of the treatment device). For example, the LEDs may be at the tips of the treatment features or on the bar member pointed at an angle outward from the device, or in any other location, arrangement, and/or orientation so as to direct light onto the skin surface. The LEDs may be aimed in front of, to the side, or between the treatment elements so that the lights may be incident skin of a user prior to and/or after the treatment elements pass a region of skin of a user. In an alternative embodiment, LEDs may additionally or alternatively be disposed on the bar, optionally facing the same direction as the treatment elements so that the LEDs may illuminate skin during use. In an embodiment, a motion sensor may sense when the device is in use (e.g., moving back and forth) and cause the LEDs to automatically turn ON during motion, and cause the LEDs to automatically turn OFF when not in motion or not moving in a particular treatment motion (e.g., substantially linearly forward/backward or side-to-side, where substantially means that there may be some rotational movement during operation). One or more pressure sensors (e.g., between the treatment element(s) and bar) may also be utilized to determine when the device is in operation and cause the LEDs to turn ON and OFF. A timing circuit may be utilized to maintain the LEDs in the ON state for a minimum duration of time (e.g., 15 seconds). In an embodiment, circuitry may turn the LEDs ON and OFF in a particular pattern, such as lighting certain LEDs when moving in a first direction and other LEDs when moving in a second direction.
A soft tissue stimulation system configured to provide electrical current to the treatment area by placing a plurality of electrodes on the skin surface and providing electrical impulses via the electrodes to the skin and soft tissue (such as fascia tissue) below the skin's surface. In some embodiments, the stimulation system employs circuitry and hardware elements that can execute traditional TENS (transcutaneous electrical nerve stimulation) and/or LAMES (neuromuscular electrical stimulation) therapy. In such embodiments, at least one lead wire may be electrically coupled to the device, with an transcutaneous electrode at the distal end for delivering the electrical impulses to the patient. The transcutaneous electrode may adhere to the skin.
   The device may be configured to provide a pre-determined stimulation waveform having a pre-determined frequency (Hz), pulse width (μs), and amplitude (mA). Alternatively the device may be configured to allow a user to modify one or more parameters of the stimulation waveform.
   The electrodes may alternatively be positioned on the device body (such as on the frame, the bar member, the treatment elements, or the treatment accessory, to be placed in direct contact with the skin for stimulation.
   In an embodiment, the treatment accessories 112, for example, may be configured with an accessible compartment (not shown) that is configured to store batteries, control circuitry, electrode(s), wires, etc., thereby enabling the treatment to be self-contained within the treatment accessories 112. See, for example, FIG. 19. User input controls, such as knobs, buttons, or otherwise, may be accessible to a user to control operation of the stimulation signals applied to a user from the electrodes controlled by the control circuitry. In operation, the user may remove the electrode(s) from the compartment and apply to him or herself. The electrodes, in an embodiment, may be attached to straps that may wrap around or be applied to a person's body, such as an arm or leg, so as to apply the TENS or NMES treatment before, during, or after fascia tissue treatment by the device with the treatment elements. As an example, the electrodes may be positioned to the sides of a pathway that the treatment elements are to be applied and electrical stimulation may occur before, during, or after treatment.

The previous description is of at least one embodiment for implementing the invention, and the scope of the invention should not necessarily be limited by this description.

What is claimed is:

1. A fascia tissue treatment device, comprising:
a bar member;
at least one tissue treatment element supported by the bar member; and
at least one treatment accessory removably connected to the bar member.

2. The fascia tissue treatment device according to claim 1, wherein the at least one treatment accessory includes a brush.

3. The fascia tissue treatment device according to claim 1, wherein the at least one treatment accessory includes a battery, one or more LEDs, and a control circuit for powering and controlling the one or more LEDs.

4. The fascia tissue treatment device according to claim 1, wherein the at least one treatment accessory includes a battery, one or more electrodes, and a control circuit for applying stimulation signals to the one or more electrodes while in use.
